(19) United States
(12) Patent Application Publication
(10) Pub. No.: US 2024/0304298 A1
(43) Pub. Date: Sep. 12, 2024

(54) Data Collection Apparatus for Dose Determination

(71) Applicant: Sanofi, Paris (FR)

(72) Inventors: Michael Schabbach, Frankfurt am Main (DE); Victor Zhitomirski, Cambridge (GB); Horst Mischo, Frankfurt am Main (DE)

(21) Appl. No.: 18/574,891

(22) PCT Filed: Jul. 9, 2021
(86) PCT No.: PCT/EP2021/069124

§ 371 Date: Dec. 28, 2023

(51) Int. Cl. G16H 20/17; A61M 5/315; G06F 3/044

(52) U.S. Cl. CPC G16H 20/17; A61M 5/31551; G06F 3/044; A61M 2205/0244; A61M 2205/3365; A61M 2205/3576; A61M 2205/50

(57) ABSTRACT

A data collection apparatus for measuring a dose of medicament is described. The data collection apparatus includes a gyroscope configured to detect rotation of a dose dialling component of a drug delivery device during at least one of a dose dialling operation or a dose dispensing operation and output a signal corresponding to the amount of rotation; and a mode sensing arrangement configured to output a signal indicative of whether the dose dialling operation or the dose dispensing operation is being performed on the drug delivery device.

### CROSS REFERENCE TO RELATED APPLICATIONS

The present application is the national stage entry of International Patent Application No. PCT/EP2021/069124, filed on Jul. 9, 2021, the disclosure of which is incorporated herein by reference.

### TECHNICAL FIELD

The present disclosure relates to data collection apparatus for determining a dose of medicament, such as a dose dialed into a drug delivery device or a dose dispensed from a drug delivery device.

### BACKGROUND

A variety of diseases exists that require regular treatment by injection of a medicament. Such injection can be performed by using injection devices, which are applied either by medical personnel or by patients themselves. As an example, type-1 and type-2 diabetes can be treated by patients themselves by injection of insulin doses, for example once or several times per day. For instance, a pre-filled disposable insulin pen can be used as an injection device. Alternatively, a re-usable pen may be used. A re-usable pen allows replacement of an empty medicament cartridge by a new one. Either pen may come with a set of one-way needles that are replaced before each use. The insulin dose to be injected can then for instance be manually selected at the insulin pen by turning a dosage knob and observing the actual dose from a dose window or display of the insulin pen. The dose is then injected by inserting the needle into a suited skin portion and pressing the dosage knob or an injection button of the insulin pen. To be able to monitor insulin injection, for instance to prevent false handling of the insulin pen or to keep track of the doses already applied, it is desirable to measure information related to a condition and/or use of the injection device, such as for instance information on the injected insulin dose.

In some previous injection devices with electronic monitoring means, the inclusion of the electronic monitoring means requires substantial modification of the injection device from standard injection device designs. Such substantial modification can result in a modified injection device that is cumbersome, expensive and difficult to manufacture.

### SUMMARY

According to an aspect of the present disclosure, there is provided a data collection apparatus for measuring a dose of medicament, the apparatus comprising: a gyroscope configured to detect rotation of a dose dialling component of a drug delivery device during at least one of a dose dialling operation and a dose dispensing operation and output a signal corresponding to the amount of rotation; and a mode sensing arrangement configured to output a signal indicative of whether the dose dialling operation or the dose dispensing operation is being performed on the drug delivery device. Such an apparatus can provide a simple, compact means for determining a dose of medicament associated with a drug delivery means. The mode sensing arrangement allows the apparatus to distinguish between rotation of the device due to an actual dose dialling and/or dispensing operation and rotation of the device due to mere handling or transportation of the device not associated with a dose dialling and/or dispensing operation.

The data collection apparatus may be configured to determine the dose based at least in part on the signal output by the gyroscope and the signal output by the mode sensing arrangement.

The dose may be a dispensed dose and the apparatus may be configured to determine the dispensed dose based on signals output from the gyroscope while the mode sensing arrangement was outputting the signal indicating that the dose dispensing operation was taking place. This may improve the accuracy of the determined dispensed dose, by ensuring the output of the gyroscope is due to an intentional dose dispensing operation.

The mode sensing arrangement may comprise an accelerometer, wherein the data collection apparatus is configured to determine the dose based on a dose determined using the gyroscope and a dose determined using the accelerometer. This may provide for a more accurate determination of the dialled or dispensed dose, by reducing the effect of possible errors in a dose determined using either of the accelerometer or the gyroscope alone.

The mode sensing arrangement may comprise an accelerometer configured to detect vibrations during the dose dialling operation or dose dispensing operation.

The accelerometer may be configured to detect vibrations of the dose dialling component.

The data collection apparatus may be configured to process a signal output by the accelerometer to distinguish between a first type of vibration and a second type of vibration, wherein the first type of vibration corresponds to the dose dialling operation and the second type of vibration corresponds to the dose dispensing operation. This may provide an effective way to determine whether it is the dose dialling operation or the dose dispensing operation is being performed on the drug delivery device.

The mode sensing arrangement may comprise an accelerometer configured to detect vibrations of the dose dialling component during the dose dialling operation or dose dispensing operation. This provides a simple means of detecting a dose dialling and/or dose dispensing operation on the drug delivery device which requires little or no modification of a standard drug delivery device.

At least one of the gyroscope and the accelerometer may be microelectromechanical systems (MEMS). This provides a simple, compact and cost effective means for dose measurement. This is particularly the case if both the gyroscope and the accelerometer are MEMS in the same chip package (e.g. a six or nine axis motion sensor).

The data collection apparatus may further comprise a communication interface, wherein the apparatus is configured to use the communication interface to transmit data corresponding to the signal output by the gyroscope to a computing device for determining the dose. This may allow for some or all of the processing of the output data to be performed away from the apparatus and on the computing device. This may reduce the processing requirements of the apparatus, allowing it to be cheaper, more simple to manufacture, and reducing the power requirements of the apparatus.

The accelerometer may be configured to detect linear translation of the dose dialling component during a dose dispensing operation. This may be a simple manner of detecting a dose dispensing operation that requires little or no modification of a standard drug delivery device.

The mode sensing arrangement may comprise an actuation sensor configured to detect that the dose dispensing operation is being performed. This may be a simple manner of detecting a dose dispensing operation that requires little or no modification of a standard drug delivery device.

The actuation sensor may comprise a capacitive touch sensor configured to detect a user's finger during the dose dispensing operation. This may be a simple manner of detecting a dose dispensing operation that requires little or no modification of a standard drug delivery device.

Furthermore, the sue of a capacitive sensor may allow the apparatus to distinguish between actuation of part of the drug delivery device by a user (i.e. actuation by a human's finger) and an object (e.g. accidental actuation within a carrying case of the drug delivery device).

The drug delivery device may be an injection pen.

The apparatus may be contained within the drug delivery device.

The apparatus may be a supplementary device attachable to the drug delivery device. This may allow the high specification and/or expensive components to be contained in the supplementary device, which can then be attached to a relatively low-cost drug delivery device. This may be helpful if the supplementary device is to be used with more than one drug delivery device.

According to another aspect of the present disclosure, there is provided a system comprising: a data collection apparatus according to any preceding disclosure; and a computing device, wherein the computing device is configured to determine the dose based on the signal output by the gyroscope and the signal output by the mode sensing arrangement.

According to another aspect of the present disclosure, there is provided a computer-implemented method for measuring a dose of medicament using, the method comprising: detecting, using a gyroscope, the rotation of a dose dialling component of a drug delivery device; determining, using a mode sensing arrangement, that a dose dialling operation or a dose dispensing operation is being performed on the drug delivery device; and in response determining that the dose dialling operation or the dose dispensing operation is being performed on the drug delivery device, determining a dose based on the rotation of the gyroscope. This can provide a simple manner for determining a dose of medicament associated with a drug delivery device.

Determining that a dose dialling operation or a dose dispensing operation is being performed on the drug delivery device may comprise: detecting vibrations of the dose dialling component using an accelerometer of the mode sensing arrangement; and determining that the detected vibrations are indicative of a dose dialling operation or a dose dispensing operation being performed on the drug delivery device.

Determining that a dose dispensing operation is being performed on the drug delivery device may comprise: detecting actuation of the dose dialling component using an actuation sensor of the mode sensing arrangement.

According to another aspect of the present disclosure, there is provided a data collection apparatus for measuring a dispensed dose of medicament, the apparatus comprising: a gyroscope configured to detect rotation of a component of a drug delivery device during a dose dispensing operation and output a signal corresponding to the amount of rotation; and a mode sensing arrangement configured to output a signal indicative of whether the dose dispensing operation is being performed on the drug delivery device, wherein the apparatus is configured to determine the dispensed dose based on signals output from the gyroscope while the mode sensing arrangement was outputting the signal indicating that the dose dispensing operation was taking place.

### BRIEF DESCRIPTION OF THE FIGURES

Exemplary embodiments will now be described with reference to the accompanying figures, of which:

FIG. 1 is a block diagram illustrating components of an apparatus according to embodiments of the disclosure;

FIG. 2 shows a side view of a drug delivery device containing the apparatus of FIG. 1, according to embodiments of the disclosure;

FIG. 3 shows the apparatus of FIG. 1 contained in a supplementary device attached to part of a drug delivery device, according to embodiments of the disclosure;

FIG. 4 shows a system according to embodiments of the invention comprising the drug delivery device and apparatus of FIG. 2 and a computing device, during a dose dialling operation;

FIG. 5 shows the system of FIG. 4 during a dose dispensing operation, according to embodiments of the disclosure; and

FIG. 6 is a flowchart illustrating a method for determining a dialled dose using the apparatus of FIG. 1, according to embodiments of the disclosure;

FIG. 7 is a flowchart illustrating a method for determining a dispensed dose using the apparatus of FIG. 1, according to embodiments of the disclosure;

FIG. 8 is a flowchart illustrating a method for determining a dispensed dose using the apparatus of FIG. 1, according to embodiments of the disclosure; and

FIG. 9 is a flowchart illustrating a method for determining a dispensed dose using the apparatus of FIG. 1, according to embodiments of the disclosure.

### DETAILED DESCRIPTION

In the following, embodiments of the present disclosure may be described with reference to a drug delivery device in the form of an insulin injection device. The present disclosure is however not limited to such application and may equally well be deployed with injection devices that eject other medicaments, or to other forms of drug delivery device. Like reference numerals refer to like elements throughout.

Aspects of the disclosure relate to determining a dose of medicament using a gyroscope 102 and a mode sensing arrangement 104.

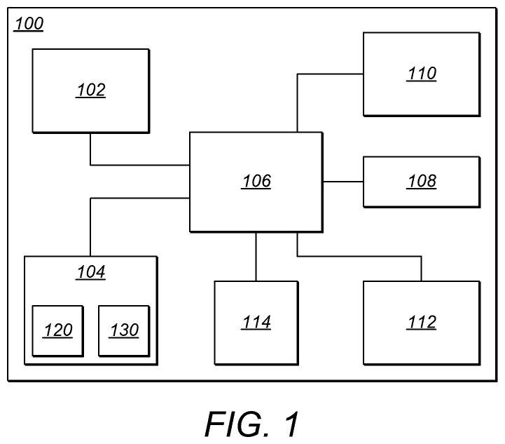

FIG. 1 is a block diagram illustrating the core components of the concept. FIG. 1 shows a data collection apparatus 100 according to the present disclosure. The data collection apparatus 100 is used for determining a dose of medicament associated with a drug delivery device 200. The dose of medicament associated with the drug delivery device 200 may be, for example, a dose that has been dialled (programmed) into the drug delivery device 200 (a ‘dialled dose’) or a dose dispensed from the drug delivery device 200 (a ‘dispensed dose’).

The apparatus 100 comprises an gyroscope 102. The gyroscope 102 is an electronic gyroscope 102 configured to output an electrical signal corresponding to the angular velocity of the gyroscope 102, and hence corresponding to the amount of rotation of the gyroscope 102 about an axis. The signal corresponding to the angular velocity that is output by the gyroscope 102 may be processed to determine an angle of rotation of the gyroscope 102, for example by integrating the angular velocity over time. This may be processed by a processor 106 of the apparatus 100 or by a processor of another device, as described later.

The gyroscope 102 may be a three-axis gyroscope 102 which can measure the angular velocity of the gyroscope 102 about three axes, such as the x-axis, y-axis and z-axis, and output a signal corresponding to the angular velocity about each axis. The angle of rotation of the gyroscope 102 in three-dimensional space may therefore be calculated from the signal output by the gyroscope, which can be used to determine the orientation of the gyroscope 102 in the three-dimensional space. However, the gyroscope 102 is not limited to a three-axis gyroscope 102 and may instead may be a one-axis gyroscope 102, a two-axis gyroscope 102, or a gyroscope 102 that can detect rotation about a higher number of axes.

The gyroscope 102 may be microelectromechanical systems (MEMS) gyroscope 102 and may be comprised in a chip package, also known as an integrated circuit package. This provides a particularly compact incarnation of the gyroscope 102.

The apparatus 100 also has a mode sensing arrangement 104. The mode sensing arrangement 104 is used to determine whether a particular operation is being performed by a user on the drug delivery device 200.

The particular operation may be a dose dialling operation. A dose dialling operation is an operation during which a dose of medicament is dialled (programmed) into the drug delivery device 200 by a user, in preparation for dispensing. The dose dialled into the drug delivery device 200 during the dose dialling operation may be known as a dialled dose.

Additionally or alternatively, the particular operation may be a dose dispensing operation. A dose dispensing operation is an operation during which a dose of medicament is dispensed from the drug delivery device 200. The dose dispensed from the drug delivery device 200 during the dose dispensing operation may be known as a dispensed dose. The dispensed dose may be equal in size to the dialled dose, or it may be smaller than the dialled dose if less than the entire dialled dose is dispensed. The dose dialling operation and dose dispensing operation are described in more detail later.

The mode sensing arrangement 104 can be used to determine whether the particular operation is currently being carried out at a particular instance in time. For example, the mode sensing arrangement 104 may be used to determine whether the dose dialling operation is currently being performed by a user at a particular instance in time, or whether the dose dispensing operation is currently being performed by a user at a particular instance in time.

The mode sensing arrangement 104 may output a signal corresponding to whether the particular operation is currently being performed. The output signal will be different when the particular operation is being performed compared to when it is not being performed. In other words, a first signal may be output when the particular operation is being performed and a second signal is output when the particular operation is not being performed. The signal output by the mode sensing apparatus 104 may be processed to determine whether or not the particular operation is being performed at a given moment in time, and in some cases for how long the operation is being performed. The signal may be processed by a processor 106 of the apparatus 100 or by a processor of another device, as described later.

The mode sensing arrangement 104 may include at least one of an accelerometer 120 and an actuation sensor 130.

The accelerometer 104 may be a MEMS accelerometer 120 and may be comprised in a chip package. The accelerometer 104 may be comprised in the same chip package as the gyroscope 102. Such a chip package containing a three-axis gyroscope 102 and three-axis accelerometer 120 is commonly known as a six-axis motion sensor. Providing the gyroscope 102 and the accelerometer 120 together in a six-axis motion sensor can allow for a particularly compact arrangement of the apparatus 100 overall. An alternative chip package is a nine-axis motion sensor, which includes a three-axis magnetometer in addition to a three-axis gyroscope 102 and a three-axis accelerometer 120.

The accelerometer 120 may be a three-axis accelerometer 120. However, the accelerometer 120 is not limited to a three-axis accelerometer 120 and may instead may be a one-axis accelerometer 120, a two-axis accelerometer 120, or a accelerometer 120 that can detect acceleration along a higher number of axes than three.

The accelerometer 120 outputs a signal corresponding to the acceleration of the accelerometer 120 along one or more axes. If the accelerometer 120 is a three-axis accelerometer 120 then the accelerometer 120 may output a signal corresponding to the acceleration of the accelerometer 120 along each of the x-axis, y-axis and z-axis. The signal output by the accelerometer 120 may be processed to determine an acceleration and/or velocity of the accelerometer 120, or a linear distance travelled by the accelerometer 120. The signal may be processed by a processor 106 of the apparatus 100 or by a processor of another device, as described later.

The accelerometer 120 may be configured to detect vibrations, such as vibrations of the accelerometer 120 and a component to which it is attached. The accelerometer 120 may be configured to output a signal corresponding to the vibrations. This signal may be the same signal as the signal corresponding to the acceleration of the accelerometer 120. The signal output by the accelerometer 120 corresponding to the vibrations may be processed, for example by a processor 106 of the apparatus 100 or by a processor of another device, as described later. The signal corresponding to the vibrations may be processed to distinguish between different types of vibration. A first type of vibration may correspond to the dose dialling operation while a second type of vibration may correspond to the dose dispensing operation.

As discussed later, the signals output by the accelerometer 120 may be used to determine whether the dose dialling operation is being performed on the drug delivery device 200 and/or whether the dose dispensing operation is being performed on the drug delivery device 200. For example, it may be determined whether a dose dialling operation or a dose dispensing operation is currently being performed on the drug delivery device 200 based on an acceleration of the accelerometer 120, as determined from the signal output by the accelerometer 120. In some examples, it may be determined whether a dose dialling operation or a dose dispensing operation is currently being performed on the drug delivery device 200 based on the vibrations of the accelerometer 120, as represented by the signal output from the accelerometer 120.

The actuation sensor 130 is configured to provide an output signal indicative of whether a dose dispensing operation is being performed by a user. The actuation sensor 130 may detect the actuation of part of the drug delivery device 200 by a user a dose dispensing operation. The actuation is indicative of the dose dispensing operation being performed on the drug delivery device 200. The part of the drug delivery device 200 may be an injection button 11, dosage knob or dialling sleeve 70, for example, as described later.

The actuation sensor 130 may comprise a switch, a capacitive touch sensor, or any other suitable type of sensor. The arrangement of the actuation sensor 130 is discussed later in relation to various embodiments. The actuation sensor 130 may be configured to provide a first output signal in response to actuation of the part by the user, and a second, different output signal when no actuation by a user is taking place.

The apparatus 100 further comprises a processor 106 and a power source 110. The processor 106 may be of any suitable type such as a microprocessor, a Digital Signal Processor (DSP), Application Specific Integrated Circuit (ASIC), Field Programmable Gate Array (FPGA) or the like. The processor 106 may be configured to perform one or more of the method steps described herein. The apparatus 100 may also comprise one or more memories 108 for storing software and/or data. For example, one or more memories 108 may comprise one or more of a program memory which stores operating software which is executed by the processor 106, a sensor data memory which stores sensor data output by the gyroscope 102 and/or mode sensing arrangement 104, or a dose history memory which stores data on the amount and timing of past doses.

The power source 110 is configured to provide power to the other electronic components of the apparatus 100 such as the gyroscope 102, mode sensing arrangement 104 and processor 106. The power source 110 may take a number of forms. The power source 110 may comprise a battery such as a coin cell or Lithium Ion battery. Alternatively or additionally, the power source 110 may comprise an energy harvester and storage system. The energy harvester may collect energy from the operations of the drug delivery device 200, in particular from the operation of dialling a dose into the drug delivery device 200. This energy may then be stored in a suitable way, for example by compressing or tensioning a spring or by direct conversion to electrical energy by a generator and subsequent storage in a capacitor. The stored energy is then used to power the various electronic components of the apparatus 100. In some examples the power source 110 may receive power wirelessly, for example through Near Field Communication (NFC).

The apparatus 100 may further comprise a switch 114 such as a microswitch. In some examples, the switch 114 is a power switch configured to selectively supply power from the power source 110 to the other components of the apparatus 100, depending on the position of the switch 114. For example, the switch 114 may be movable between an ‘off’ position, in which components of the apparatus 100 are isolated from the power source 110, and an ‘on’ position, in which components of the apparatus 100 are electrically connected to the power source 110. The switch 114 may be operated by a user of the drug delivery device 200.

In some examples, the switch 114 is configured to provide an input to the processor 106 in response to user actuation of the switch 114. The switch 114 can therefore act as a user input to the apparatus 100, allowing the user to select or confirm an operation.

In some examples, some or all of the electronic components may operate in a very low power mode or an ‘off’ mode until the processor 106 receives a wake-up signal from the switch 114, caused by a user actuating the switch 114. In response to receiving the wake-up signal, one or more of the processor 106, the gyroscope 102 and the accelerometer 120 wake-up so that the dialled or delivered dose of medicament can be monitored. The processor 106 is configured to draw power from the power source 110 and to control the application of power to the gyroscope 102 and the mode sensing arrangement 104. The gyroscope 102 and the accelerometer 120 can then be used to monitor a dose dialled into the drug delivery device 200 and/or a dose delivered from the drug delivery device 200, as discussed later, and to output signals to the processor 106 corresponding to the amount of medicament dialled and/or delivered.

In some examples, the switch 114 may also act as the actuation sensor 130.

The apparatus 100 may further comprise a communication interface 112. The apparatus 100 is configured to transmit data using the communication interface 112 to another computing device 410 such as a smartphone, tablet or PC for processing or storage. In some cases the apparatus 100 is also configured to receive data from the computing device 410 using the communication interface 112. The communication interface 112 may be a wired or a wireless communication interface 112. An example of a wireless communication interface 112 may be a Bluetooth communication interface configured to transmit data using a Bluetooth protocol. Another example of a wireless communication interface 112 may be a Near-Field Communication (NFC) interface configured to transmit data using an NFC protocol. Other suitable types of wireless communication interface 112 and/or protocols may be used. The transmission of data via the communication interface 112 may be controlled by the processor 106. Data transmitted via the communication interface 112 may include data corresponding to one or more of the signals output by the gyroscope 102, accelerometer 120 and actuation sensor 130. Data transmitted via the communication interface 112 may include data corresponding to dose values.

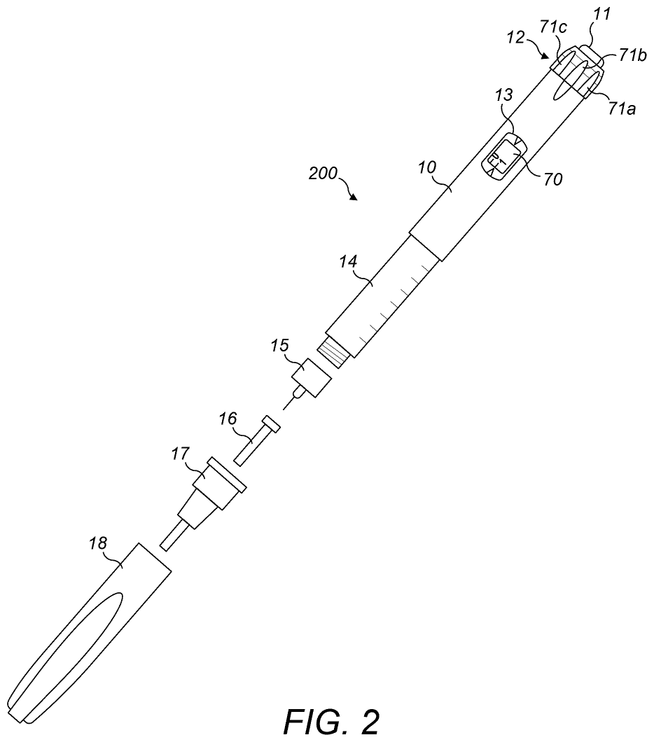

The apparatus 100 of FIG. 1 can be used to determine a dose of medicament associated with a drug delivery device 200. As will be described in detail below, the apparatus 100 of FIG. 1 may be embodied in a number of different ways. For example, the apparatus 100 may be part of the drug delivery device 200 itself, as discussed in relation to FIG. 2. The components of the apparatus 100 may be contained within the drug delivery device 200 such as within the housing 10, dosage knob 12, injection button 11 or dialling sleeve 70 of the drug delivery device, as discussed later in relation to FIG. 2.

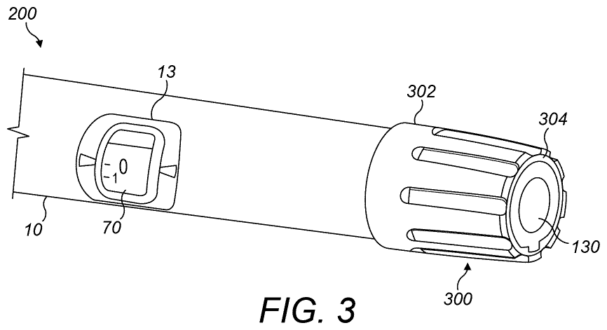

In some other embodiments, the apparatus 100 may be comprised as part of a supplementary device 300 which is configured to be secured to a drug delivery device 200, as discussed later in relation to FIG. 3. For example, the supplementary device 300 may be configured to be releasably secured to the housing 10, dosage knob 12 or injection button 11 of a drug delivery device 200.

In some further embodiments, some of the components of the apparatus 100 are part of the drug delivery device 200 while other components are part of a supplementary device 300. For example, the gyroscope 102 and/or the accelerometer 120 may be part of the supplementary device 300, while the processor 106 and power source 110 may be part of the drug delivery device 200.

FIG. 2 is an exploded view of a drug delivery device 200 containing the apparatus 100 of FIG. 1 according to embodiments. In this example, the drug delivery device 200 is an injection device, such as Sanofi's SoloSTAR® insulin injection pen, but aspects may apply to other types of injection pen or injection device. The drug delivery device 200 of FIG. 2 is a pre-filled, disposable injection pen, but in other examples the drug delivery device 200 may be reusable.

The drug delivery device 200 of FIG. 2 comprises a cylindrical housing 10 and contains an insulin container 14, to which a needle 15 can be affixed. The needle is affixed at the distal end of the housing 10. The term “distal” as used throughout refers to a location that is relatively closer to a site of injection, and the term “proximal” refers to a location that is relatively further away from the injection site. The needle 15 is protected by an inner needle cap 16 and either an outer needle cap 17 other cap 18.

During a dose dialling operation, an insulin dose to be ejected from drug delivery device 200 can be dialled, or ‘programmed in’ by turning a dosage knob 12, and a currently dialled dose is then displayed via dosage window 13, for instance in multiples of units. For example, where the drug delivery device 200 is configured to administer human insulin, the dosage may be displayed in so-called International Units (IU), wherein one IU is the biological equivalent of about 45.5 micrograms of pure crystalline insulin (1/22 mg). Other units may be employed in drug delivery devices 200 for delivering analogue insulin or other medicaments. It should be noted that the selected dose may equally well be displayed differently than as shown in the dosage window 13 in FIG. 2.

The dosage window 13 may be in the form of an aperture in the housing 10, which permits a user to view a limited portion of a dialling sleeve 70. The dialling sleeve 70 is configured to move when the dosage knob 12 is turned, to provide a visual indication of a currently dialled dose. The dialling sleeve 70 may also be known as a number sleeve. The dosage knob 12 is rotated on a helical path with respect to the housing 10 when turned during a dose dialling operation.

In this example, the dosage knob 12 includes one or more formations 71a, 71b, 71c to assist a user in gripping the dosage number 12 during dialling. The formations 71a, 71b, 71c may be grooves, ridges or the like.

The drug delivery device 200 may be configured so that turning the dosage knob 12 during the dose dialling operation causes a mechanical click or vibration sound to provide acoustical and/or haptic feedback to a user. The dialling sleeve 70 mechanically interacts with a piston in insulin container 14. When needle 15 is stuck into a skin portion of a patient, and then injection button 11 is pushed during a dose dispense operation, the insulin dose displayed in display window 13 will be ejected from drug delivery device 200. When the needle 15 of drug delivery device 200 remains for a certain time in the skin portion after the injection button 11 is pushed, a high percentage of the dose is actually injected into the patient's body. Ejection of the insulin dose may also cause a mechanical click or vibration sound, which is however different from the sounds produced when using dosage knob 12.

Drug delivery device 200 may be used for several injection processes until either the insulin container 14 is empty or the expiration date of the medicament in the drug delivery device 200 (e.g. 28 days after the first use) is reached.

Furthermore, before using drug delivery device 200 for the first time, it may be necessary to perform a so-called “prime shot” to remove air from insulin container 14 and needle 15, for instance by selecting two units of insulin and pressing injection button 11 while holding drug delivery device 200 with the needle 15 upwards. For simplicity of presentation, in the following, it will be assumed that the ejected amounts substantially correspond to the injected doses, so that, for instance the amount of medicament ejected from the drug delivery device 200 is equal to the dose received by the user. Nevertheless, differences (e.g. losses) between the ejected amounts and the injected doses may need to be taken into account.

The apparatus 100 may be contained within the drug delivery device 200, as shown in FIG. 2. For example, the apparatus 100 may be contained within the dosage knob 12, the injection button 11 or the dialling sleeve 70 of the drug delivery device 200.

The gyroscope 102 of the apparatus 100 is coupled to a rotatable dose dialling component of the drug delivery device 200. The rotatable dose dialling component is a component of the drug delivery device 200 that rotates relative to the housing 10 during a dose dialling operation, in which as a dose of medicament is dialled into the drug delivery device 200 by a user. During a dose dialling operation, the dose dialling component may also translate linearly with respect to the housing 10 of the drug delivery device 200 as a dose of medicament is dialled into the drug delivery device 200. This translation may be simultaneous to the rotation of the dose dialling component.

In some examples the rotatable dose dialling component comprises the dosage knob 12 of the drug delivery device 200, in which case the gyroscope 102 may be coupled to the dosage knob 12. In some examples the rotatable dose dialling component comprises the dialling sleeve 70, in which case the gyroscope 102 may be coupled to the dialling sleeve 70. In some examples the rotatable dose dialling component comprises the injection button 11, in which case the gyroscope 102 may be coupled to the injection button 11. However, the gyroscope 102 is not limited to being coupled to those particular rotatable dose dialling components and may be coupled to other types of rotatable dose dialling component. In some examples, the entire apparatus 100 including the gyroscope 102 is coupled to the dose dialling component.

During a dose dispensing operation, the dose dialling component may again translate linearly with respect to the housing 10, but in an opposite direction to the translation of the dose dialling component during the dose dialling operation.

The dose dialling component may in some examples also rotate relative to the housing 10 during the dose dispensing operation in a direction that is opposite to the direction of rotation during the dose dialling operation. This rotation will be simultaneous to the translation motion. However, in other examples the dose dialling component does not rotate relative to the housing 10 during the dose dispensing operation and only translates.

Part of the drug delivery device 200 such as the rotatable dose dialling component is configured to vibrate as a dose is dialled into the drug delivery device 200. The vibrations may indicate that a dose dialling operation is being performed. The vibrations may comprise a number of ‘clicks’. Each click may correspond to a predetermined number of dose units being dialled into the drug delivery device 200. For example, each click may correspond to one dose unit being dialled into the drug delivery device 200. In that example, ten clicks would therefore correspond to ten dose units being dialled. By counting the number of clicks using an accelerometer 120, a dialled dose can be determined. Each click also corresponds to a certain angle of rotation of the dose dialling component during the dose dialling operation. For example, each click may correspond to the dose dialling component being rotated eighteen degrees.

Part of the drug delivery device 200 such as the rotatable dose dialling component may be configured to vibrate as a dose is dispensed from the drug delivery device 200. The vibrations may indicate that a dose dispensing operation is being performed. The vibrations may comprise a number of ‘clicks’, with each click may corresponding to a predetermined number of dose units being dispensed from the drug delivery device 200. For example, each click may correspond to one dose unit being dispensed from the drug delivery device 200. In that example, ten clicks would therefore correspond to ten dose units being dispensed. By counting the number of clicks using an accelerometer 120, a dialled dose can be determined. If the dose dialling component rotates during a dose dispensing operation then each click may also correspond to a certain angle of rotation of the dose dialling component during the dose dispensing operation. For example, each click may correspond to the dose dialling component being rotated eighteen degrees.

The vibrations or clicks produced during the dose dialling operation may be different to the vibrations or clicks produced during the dose dispensing operation. In this way, by analysing the vibrations or clicks, it can be determined whether a dose dialling operation is being performed on the drug delivery device 200 or a dose dispensing operation is being performed on the drug delivery device 200. For example, the frequency and/or amplitude of the vibrations (or clicks) produced during a dose dialling operation may be different to the frequency and/or amplitude of the vibrations (or clicks) produced during the dose dispensing operation. In some examples, clicks will only be produced during dose dialling operations but not during dose dispensing operations.

Coupling the gyroscope 102 to the dose dialling component means that the gyroscope 102 is able to detect rotation of the dose dialling component during the dose dialling operation. The gyroscope 102 may also be able to detect rotation of the dose dialling component during the dose dispensing operation, if the dose dialling component rotates during such an operation.

During a dose dialling operation, the amount of rotation of the dose dialling component relative to the housing 10 may correspond to the size of the dose dialled into the drug delivery device 200. As such, one unit of dose may correspond to a predetermined angle of rotation of the dose dialling component relative to the housing 10. As an example, the drug delivery device 200 may be configured such that a three hundred and sixty degree rotation of the dose dialling component relative to the housing 10 corresponds to twenty units of dose being dialled into the drug delivery device 200. An eighteen degree rotation of the dose dialling component relative to the housing 10 may therefore correspond to one dose unit being dialled into the drug delivery device 200.

The dose dialled into the drug delivery device 200 may therefore be determined based on the amount of rotation of the dose dialling component detected by the gyroscope 102 during the dose dialling operation. As discussed previously, the gyroscope 102 is configured to provide an output signal indicative of the amount of rotation of the rotatable dose dialling component. The output signal may correspond to the angular velocity of the rotatable dose dialling component. The processor 106 of the apparatus 100 or a different processor of a computing device 410 or the like may determine the angular velocity from the signal output by the gyroscope 102 and integrate the angular velocity to determine an angle of rotation of the rotatable dose dialling component. The processor 106 of the apparatus 100 or the different processor may determine the dialled dose based on the determined angle of rotation, for example by dividing the determined angle by a predetermined value of dose units per unit angle rotation.

Similarly, if the dose dialling component also rotates relative to the housing 10 during a dose dispensing operation, the amount of rotation of the dose dialling component may correspond to the size of the dose dispensed by the drug delivery device 200. Therefore the dose dispensed by the drug delivery device 200 may be determined based on the amount of rotation of the dose dialling component detected by the gyroscope 102 during the dose dispensing operation, in a similar manner to determining the dialled dose.

A mode sensing arrangement 104 is also provided in the apparatus 100. If the apparatus 100 is configured to be used for determining a dose that has been dialled into the drug delivery device 200, then the mode sensing arrangement 104 will comprise an accelerometer 120 as described previously. The accelerometer 120 will be configured to detect the vibrations of the drug delivery device 200 during the dose dialling operation. The accelerometer 120 is arranged in the drug delivery device 200 such that it is able to detect the vibrations. The accelerometer 120 will be configured to output a signal corresponding to the vibrations produced during the dose dialling operation, for example the clicks produced during the dose dialling operation. The signal may be processed by the processor 106 of the apparatus 100 or a processor of a different computing device 410.

If the apparatus 100 is configured to be used for determining a dose that has been dispensed from the drug delivery device 200, then the mode sensing arrangement 104 will comprise at least one of an accelerometer 120 and an actuation sensor 130. If the apparatus the mode sensing arrangement 104 comprises an accelerometer 120 then the accelerometer may be configured to detect the vibrations of the drug delivery device 200 during the dose dispensing operation. The accelerometer 120 is arranged in the drug delivery device 200 such that it is able to detect the vibrations. The accelerometer 120 will be configured to output a signal corresponding to the vibrations produced during the dose dispensing operation, for example the clicks produced during the dose dispensing operation. The signal may be processed by the processor 106 of the apparatus 100 or a processor of a different computing device 410.

The accelerometer 120 may additionally or alternatively be arranged to output a signal corresponding to the linear translation of the dose dialling component relative to the housing 10 during a dose dispensing operation.

If the apparatus 100 comprises an actuation sensor 130 as discussed in relation to FIG. 1 then it will be arranged in the drug delivery device 200 such that it may detect the actuation of part of the drug delivery device 200 by a user during the dose dispensing operation. The actuation sensor 130 may, for example, be located in the drug delivery device 200 so that it can detect actuation of at least one of the injection button 11, dosage knob or dialling sleeve 70. If the actuation sensor 130 comprises a capacitive touch sensor then it may be arranged on an outer surface of the injection button 11.

In other examples, the apparatus 100 may be comprised in a supplementary 300 device that is attachable to a drug delivery device 200, as shown in FIG. 3.

FIG. 3 shows a supplementary device 300 according to another embodiment, where the supplementary device 300 comprises the apparatus 100. The supplementary device 300 is configured to be releasably attached to a drug delivery device 200 such as the drug delivery device 200 shown in FIG. 2. The supplementary device 300 has a main body 302 which retains some or all of the components of the apparatus 100. The supplementary device 300 has a coupling arrangement (not shown) for coupling the supplementary device 300 to the rotatable dose dialling component of the drug delivery device 200, such as the dosage knob 12 or injection button 11. The supplementary device 300 may couple to the drug delivery device 200 using any suitable coupling means such as friction fit, magnets, adhesive, ridges and grooves, a screw coupling, a bayonet coupling, a latch, or bumps and recesses.

The main body 302 of the supplementary device 300 may be shaped so as to allow it to be releasably secured to the dosage knob 12 of the drug delivery device. For example, the main body 302 may have a cylindrical or truncated conical shape, with an open end for receiving the dosage knob 12 and a closed end. The supplementary device 300 may therefore effectively replace the dosage knob 12 of the drug delivery device when attached, as illustrated in FIG. 3.

In some other embodiments, the supplementary device 300 has a different form and may be configured to couple to a different part of the drug delivery device, for example to the injection button 11.

A user may be able to attach and remove the supplementary device 300 from the drug delivery device 200. The supplementary device 300 may be compatible with a number of different models of drug delivery devices 200.

The apparatus 100 of the supplementary device 300 shown in FIG. 3 operates in a similar manner to the apparatus 100 described in relation to FIG. 1 and FIG. 2 and may have similar components. When the supplementary device 300 is coupled to the dose dialling component of a drug delivery device 200, the gyroscope 102 will also be coupled to the dose dialling component of the drug delivery device 200 and so will be able to detect rotation of the dose dialling component for determining a dialled dose as discussed previously.

Similarly, when the supplementary device 300 is coupled to the dose dialling component of a drug delivery device 200, the mode sensing arrangement 104 will be able to perform in a similar manner as discussed in relation to FIG. 1 and FIG. 2. For example, if the supplementary device 300 comprises an accelerometer 120, the accelerometer 120 will be able to detect accelerations/vibrations caused by a dose dialling operation and/or dose dispensing operation as discussed previously.

The mode sensing arrangement 104 of the supplementary device 300 may comprise an actuation sensor 130 as discussed previously. FIG. 3 shows the actuation sensor 130 arranged at an outer surface 304 of the supplementary device 300. The actuation sensor 130 is located such that it is actuated by a user when performing a dose dispensing operation. FIG. 3 shows the supplementary device 300 coupled to the dosage knob 12 or injection button 11 of the drug delivery device 200. When a user wishes to perform a dose dispensing operation they apply a force to the injection button 11 or dosage knob 12 via the supplementary device 300. This force is detected by the actuation sensor 130.

The actuation sensor 130 may take the form of a capacitive touch sensor arranged at the outer surface 304 of the supplementary device 300, which can detect a user's finger pressing the supplementary device 300. Using a capacitive touch sensor as the actuation sensor 140 can allow the apparatus 100, or another device connected to the apparatus 100, to discriminate between a ‘real’, intended actuation of the injection button 11 by the finger of a user and a ‘false’ actuation by something else such as a storage container used to contain the drug delivery device 200.

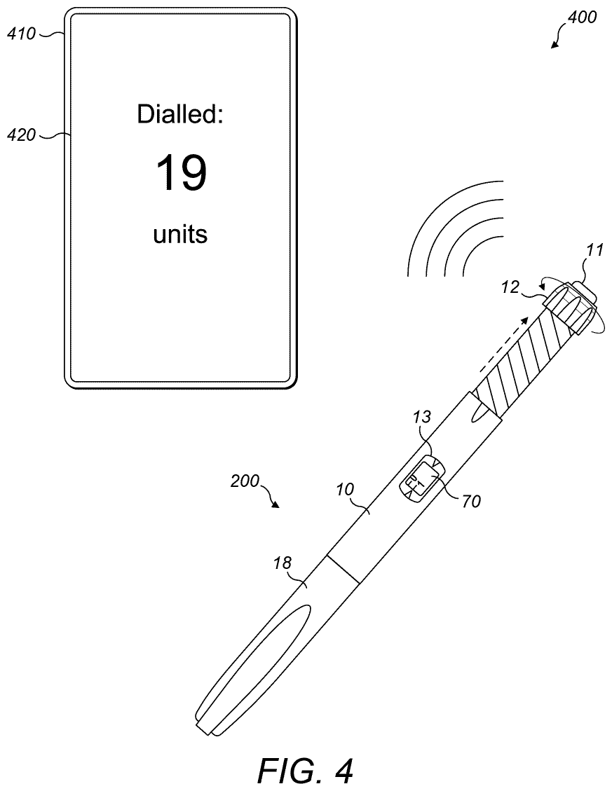

Aspects of the present disclosure also relate to a system 400 as illustrated in FIG. 4. FIG. 4 shows the system 400 comprising a drug delivery device 200 and an apparatus 100 as discussed previously, along with a computing device 410. The drug delivery device 200 and apparatus 100 are similar to the drug delivery device 200 and apparatus 100 previously shown in FIG. 2. The apparatus 100 is contained within the dosage knob 12 of the drug delivery device 200 and so is not visible in FIG. 4. However, the system 400 may alternatively include other arrangements of the apparatus 100 as described previously. For example, the apparatus 100 may instead be contained in the injection button 11 or the dialling sleeve 70 of the drug delivery device 200. Alternatively, the apparatus 100 may be comprised in a supplementary device 300 such as the supplementary device shown in FIG. 3, and coupled to a dose dialling component of a drug delivery device 200 such as the dosage knob 12, injection button 11 or the dialling sleeve 70.

FIG. 4 illustrates a dose dialling operation taking place, in which a user dials a dose into the drug delivery device 200 ready for ejection. During the dose dialling operation, the user holds the housing 10 of the drug delivery device 200 with one hand and with the other hand rotates the dosage knob 12 of the drug delivery device 200 relative to the housing 10. The rotation of the dosage knob 12 relative to the housing 10 is indicated by the curved arrow around the dosage knob 12. The dialling sleeve 70 and injection button 11 also rotate together with the dosage knob 12 as the dose is dialled into the drug delivery device 200. The number of units displayed through the dosage window 13 increases as the dose dialled into the drug delivery device 200 increases. The rotation of the dosage knob 12 is detected by the gyroscope 102 of the apparatus 100 which outputs a corresponding signal, as discussed previously.

As the dosage knob 12, dialling sleeve 70 and injection button 11 are rotated during the dose dialling operation, they also translate linearly away from the housing 10, as indicated by the dotted arrow. The drug delivery device 200 is configured to vibrate during the dose dialling operation, as discussed previously. The vibrations (or clicks) are detected by the accelerometer 120, which outputs a corresponding signal.

In some cases a user may also decrease the dose dialled into the drug delivery device 200 by rotating the dosage knob 12 in the opposite direction, causing the dosage knob 12 to move back towards the housing 10. The rotation of the dosage knob 12 in the opposite direction can also be detected by the gyroscope 102. The accelerometer 120 may also detect vibration of the drug delivery device 200 as the dosage knob 12 is rotated back.

FIG. 4 also shows the system 400 including a computing device 410 such as a smartphone, tablet or PC. The computing device 410 is external to the apparatus 100 and drug delivery device 200. Like the apparatus 100, the computing device 410 may have a processor for performing processing/determining operations, as well one or more memories and a power source (not shown). The computing device 410 also has a communication interface (not shown) for communicating with the communication interface 112 of the apparatus 100 wirelessly or in a wired fashion. For example, the computing device 410 and apparatus 100 may communicate using Bluetooth or NFC.

The apparatus 100 may be configured to transmit data to the computing device 410 via its communication interface 112. The data may be data corresponding to the signals output by the gyroscope 102, the accelerometer 120, or other components of the apparatus 100 such as the actuation sensor 130. For example, the apparatus 100 may be configured to transmit data corresponding to amount rotation of the dose dialling component as detected by the gyroscope 102. The apparatus 100 may be configured to transmit data corresponding to vibrations or number of clicks as detected by the accelerometer 120.

FIG. 4 shows the computing device 410 having a display 420. The display 410 may be any type of suitable display such as an LCD display or an LED display. The display 420 may be a touch display which can also receive touch input from the user. The display 420 may be controlled by the computing device 410 to output information to a user. The information may include a representation of a dose dialled into the drug delivery device 200 as determined using the apparatus 100. For example, the display 420 may show the number of units currently dialled into the drug delivery device 200, as illustrated in FIG. 4. The information on the display 420 may be updated as the dialled dose determined using the apparatus 100 changes. For example, the dose shown on the display 420 may increase as the dose dialled into the drug delivery device 200 increases.

The dialled dose may be determined by the computing device 410 based on the data sent to the computing device 410 by the apparatus 100 such as the gyroscope 102 data and accelerometer 120 data, or it may be determined by the apparatus 100 itself and later transmitted to the computing device 410 for display/storage.

Once the user has dialled the desired dose of medicament into the drug delivery device 200, they are now ready to perform a dose dispensing operation to eject the dose.

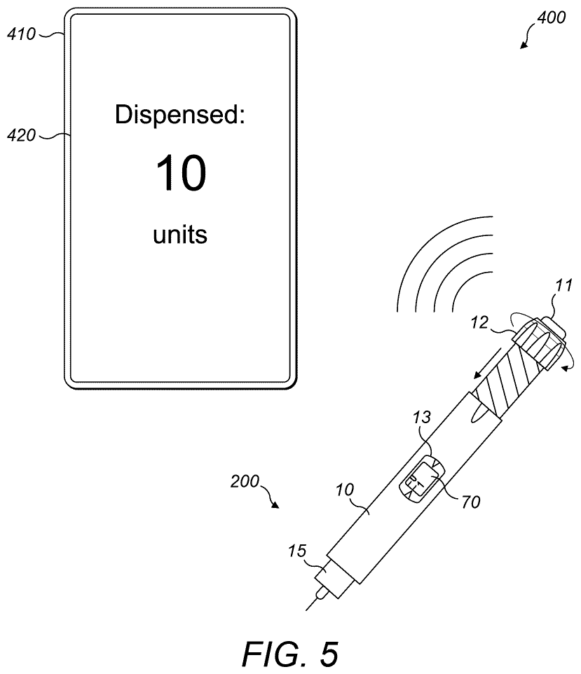

FIG. 5 shows the system 400 of FIG. 4, but now during a dose dispensing operation. The dose dispensing operation illustrated by FIG. 5 will usually be performed after the dose dialling operation illustrated by FIG. 4. However, in some cases a dose dialling operation does not need to be performed by a user. This may be the case where the size of the dose to be dispensed from the drug delivery device 200 has already been set, for example in the case of the drug delivery device 200 being an autoinjector configured to dispensed a predetermined dose of medicament.

The cap 18 has been detached from the housing 10 of the drug delivery device 200 to expose the needle 15, which may then be inserted into a patient. During the dose dispensing operation, the user actuates the injection button 11 by pressing it towards the housing 10 of the drug delivery device 200 to dispense the dose. If the apparatus 100 forms part of a supplementary device 300 coupled to the injection button 11 or dosage knob 12 then the user may push the supplementary device 300 towards the housing 10, rather than touching the injection button 11 or dosage knob 12 directly, which in turn causes the injection button 11 or dosage knob 12 to be actuated towards the housing 10. As the injection button 11 linearly translates towards the housing 10, the dosage knob 12 and dialling sleeve 70 also translate linearly with the injection button 11, as indicated by the dotted arrow. In some examples the dosage knob 12 rotates relative to the housing 10 as the injection button 11, dosage knob 12, dialling sleeve 70 linearly translate towards the housing 10, as indicated by the curved arrow. In this case, the dosage knob 12 may rotate in the opposite direction to which it rotated during dose dialling. However, in other examples the dosage knob 12 does not rotate relative to the housing 10 during the dose dispensing operation and instead only translates linearly.

The number of units displayed in the dosage window 13 may decrease as the injection button 11 is actuated and medicament is expelled from the drug delivery device 200. Medicament may be ejected from the drug delivery device 200 for as long as the user actuates the injection button 11. Medicament may stop being ejected once the user releases the injection button 11 or once the dosage knob 12, injection button 11 and dialling sleeve 70 are back in their original positions relative to the housing 11, as shown in FIG. 2. In other examples, the user may only have to press and release the injection button 11 once and the drug delivery device 200 will automatically continue to eject a predetermined amount of dose. This maybe the case if the drug delivery device 200 is an autoinjector.

If the dosage knob 12 is configured to rotate relative to the housing 10 during the dose dispensing operation then this rotation will be detected by the gyroscope 102, which will output a corresponding signal as discussed previously. The signal may be processed to determine a dispensed dose.

If the apparatus comprises an accelerometer 120 then it may detect acceleration of the dosage knob 12 towards the housing 10 during the dose dispensing operation and output a corresponding signal. This signal may be indicative that a dose dispensing operation being performed.

If the apparatus comprises an accelerometer 120 then it may detect vibrations or clicks of the dosage knob 12 or another part of the drug delivery device 200 during the dose dispensing operation and output a corresponding signal as discussed previously. This signal may be indicative that the dose dispensing operation being performed. The signal may be processed to determine a dispensed dose.

If the apparatus 100 comprises an actuation sensor 130 then actuation of the sensor 130 during the dose dispensing operation may be detected and output as a signal for processing.

Data corresponding to the signals output by the gyroscope 102, the accelerometer 120, or actuation sensor 130 may be transmitted from the apparatus 100 to the computing device 410 for processing, to determine a dispensed dose. However in some examples, determining a dispensed dose is performed by the apparatus 100, with the result then transmitted to the computing device 410.

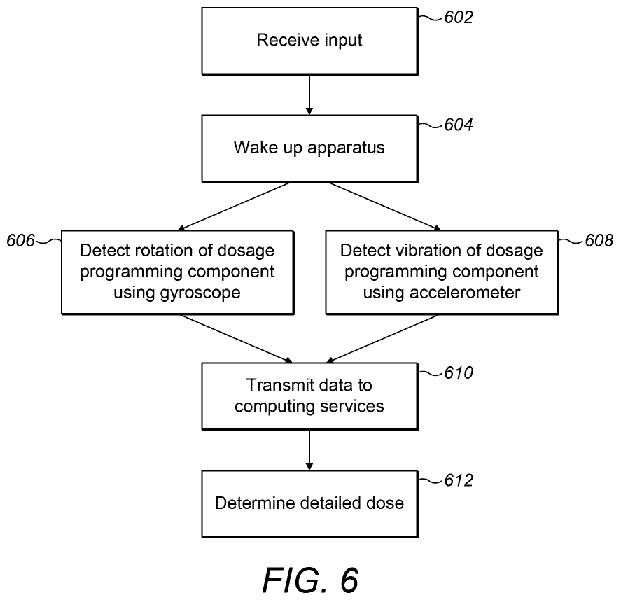

FIG. 6 is a flowchart illustrating a method of determining a dose dialled into a drug delivery device 200 using the apparatus 100, according to aspects of the disclosure. The apparatus 100 may be contained within a drug delivery device 200, for example as shown in FIG. 2, or may be part of a supplemental device 300 coupled to a drug delivery device 200, as shown in FIG. 3. The apparatus 100 and drug delivery device 200 may be part of a system 400 such as the system of FIG. 4.

In optional step 602, an user input is received at the apparatus 100. This input may be received from the switch 114 or the actuation sensor 130, such as a capacitive touch sensor.

In optional step 604, the apparatus 100 is woken up in response to receipt of the user input. For example, the processor 106 may receive an input signal from the switch 114 or the actuation sensor 130 and in response to the input signal may cause the gyroscope 102 and/or the accelerometer 120 to begin taking measurements.

In step 606, rotation of the dosage dialling component during a dose dialling operation is detected using the gyroscope 102. The gyroscope 102 outputs an electrical signal corresponding to the rotation of the dose dialling component, as discussed previously. Simultaneous to the detection of the rotation by the gyroscope 102, in step 608 the mode sensing arrangement 104 detects whether a dose dialling operation is being performed by a user on the drug delivery device. Step 608 is performed using the accelerometer 120, which detects vibration of the dose dialling component or another part of the drug delivery device 200 as the dose is dialled into the drug delivery device, as discussed previously. The accelerometer 120 outputs an electrical signal corresponding to the vibrations of the dose dialling component, as discussed previously. The signal may correspond to the number of vibrational clicks produced by the drug delivery device 200, as discussed previously.

In optional step 610, data corresponding to the signal output by the gyroscope 102 and data corresponding to the signal output by the accelerometer 120 is transmitted from the apparatus 100 to the computing device 410 via the communication interface 112. The signals output by the gyroscope 102 and the accelerometer 120 may have been at least partially processed by the processor 106 of the apparatus 100 to generate the data transmitted to the computing device 410.

In step 612, a dose of medicament dialled into the drug delivery device 200 is determined based on the signal output by the gyroscope 102 and the signal output by the accelerometer 120. If step 610 was performed, then step 612 may be performed by the computing device 410, with the computing device 41 determining the dose of medicament dialled into the drug delivery device 200 based on the data corresponding to the signal output by the gyroscope 102 and the data corresponding to the signal output by the accelerometer 120 transmitted from the apparatus 100. However if step 610 was not performed, then step 612 may be performed by the apparatus 100 itself, with the processor 106 of the apparatus 100 determining the dose of medicament dialled into the drug delivery device 200 based on the signal output by the gyroscope 102 and the signal output by the accelerometer 120.

Determining the dose of medicament dialled into the drug delivery device 200 may comprise determining a dialled dosed based on the signal output by the gyroscope 102 as discussed previously, for example by integrating the angular velocity represented by the signal output by the gyroscope 102 over time. Determining the dose of medicament dialled into the drug delivery device 200 may further comprise determining a dialled dose based on the signal output by the accelerometer 120 as discussed previously, for example by counting the number of ‘clicks’ represented by the signal and converting the number of clicks to a dose. Determining the dose of medicament dialled into the drug delivery device 200 may then comprise determining a final dialled dose based on the dose determined using the gyroscope 102 and the dose determined using the accelerometer 120. The final dialled dose may be an average of the dose determined using the gyroscope 102 and the dose determined using the accelerometer 120. The final dialled dose may be the higher of the dose determined using the gyroscope 102 and the dose determined using the accelerometer 120, or the lower of the dose determined using the gyroscope 102 and the dose determined using the accelerometer 120.

After the dialled dose has been determined in step 612, information representing the determined dialled dose may be output for display to a user and/or stored. This may be performed by the apparatus 100 and/or the computing device 410. If step 612 was performed by the computing device 410 then the computing device 410 may output the information indicating the determined dialled dose on a display 420 of the computing device 410, so that a user can view the dose currently dialled into the drug delivery device 200. If step 612 was performed by the apparatus 100, the apparatus 100 may transmit data indicating the determined dialled dose to the computing device 410 so that the computing device 410 may display the information on the display 420. Information representing the dialled dose may additionally or alternatively be displayed on a display of the apparatus 100.

Steps 606-612 may be carried out continuously. That is, as the user continues to increase the dose dialled into the drug delivery device 200, the gyroscope 102 and accelerometer 120 will continue to output respective signals, with data corresponding to the signals being sent to the computing device 410. The dialled dose may be recalculated as the dose dialling component continues to be rotated and the data is received. The updated dose may then be shown on the display 420. The dose shown on the display 420 may be updated continuously so that it changes close to real-time with the dialling of a dose.

In some examples it may be determined that the dose dialling operation has ended. This may be determined in response to a user input, for example by a user actuating switch 114, actuation sensor 130, or providing an input to the computing device 410. In some examples it may be determined by the accelerometer 120, in particular by the accelerometer 120 no longer detecting vibrations indicative of the dose dialling operation. In other examples, ending of the dose dialling operation may be determined by detecting that the dialled dose has not changed by more than a threshold amount for a predetermined amount of time, such as five seconds. In other examples, it may be determined in response to the mode sensing arrangement 104 detecting that a dose dispense operation has begun.

In response to determining that the dose dialling operation has ended, a value representing the determined dialled dose may be stored. This may be stored locally in a memory of the computing device 410 or a memory of the apparatus 100. In some examples, it may be transmitted to another computing device, a server or the cloud to be stored. The computing device 410 may output a signal to a user indicating that the dose dialling operation has ended, for example an audio signal, a haptic signal, or a visual signal using the display 420.

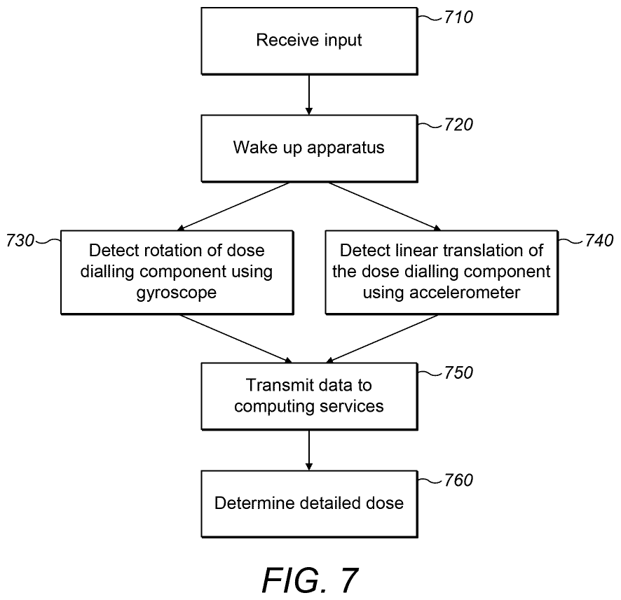

FIG. 7 is a flowchart illustrating a method of determining a dose dispensed by a drug delivery device 200 using the apparatus 100, according to aspects of the disclosure.

In some cases, the steps of FIG. 7 may follow step 612 of FIG. 6. In other cases, the apparatus 100 may only be configured to perform the method of FIG. 6 or the method of FIG. 7, but not both.

In optional step 710, the apparatus 100 receives a user input. The input may be received by the user pressing a switch 114, which may be the same switch 114 discussed in step 602. In some cases the user input may be received by a user actuating the actuation sensor 130, such as a capacitive touch sensor. A signal indicating the input has been received is transmitted from the apparatus 100 to the computing device 410.

In optional step 720, and in response to receiving the input, the apparatus may be woken up. For example, the processor 106 may receive an input signal from the switch 114 or the actuation sensor 130 and in response may causes the gyroscope 102 and the accelerometer 120 to begin taking measurements.

In step 730, the gyroscope 102 detects dispensing of a dose from the drug delivery device 200. In particular, the gyroscope 102 detects rotation of a dose dialling component as the dose is dispensed by the drug delivery device 200, and outputs a signal corresponding to the rotation of the dose dialling component, as discussed previously.

At around the same time as step 730, in step 740, linear translation of the dose dialling component relative to the housing 10 is detected by the accelerometer 120 and a signal corresponding to the linear translation of the dose dialling component is output. The linear translation is indicative of a dose dispensing operation being carried out.

In optional step 750, data corresponding to the signal output by the gyroscope 102 and the signal output by the accelerometer 120 is transmitted from the apparatus 100 to the computing device 410 via the communication interface 112. The signals output by the gyroscope 102 and the accelerometer 120 may have been at least partially processed by the processor 106 of the apparatus 100 to generate the data transmitted to the computing device 410.

In step 760, a dose dispensed by the drug delivery device 200 is determined based at least on the signal output by the gyroscope 120, in a similar manner as discussed previously. The dispensed dose may be determined by the computing device 410 or by the apparatus 100. The signal output by the accelerometer 120 may be used to confirm that a dose dispensing operation is taking place. The dispensed dose may be determined based on signals output from the gyroscope 102 while the accelerometer 120 was outputting a signal indication that a dose dispensing operation was taking place.

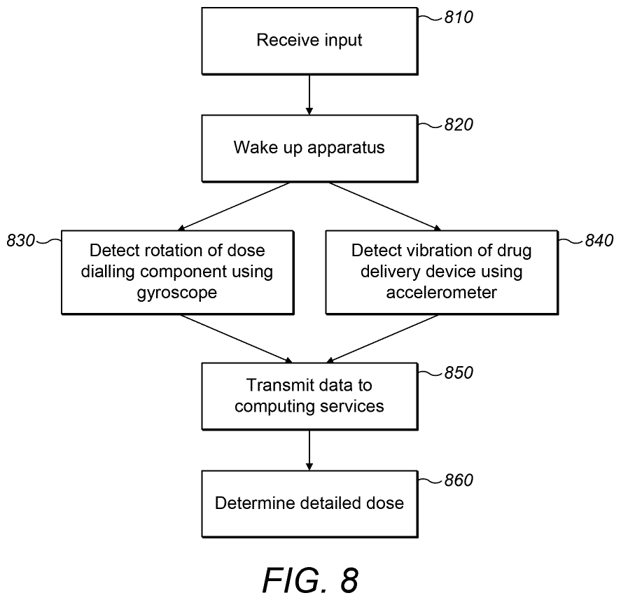

FIG. 8 shows a similar method to FIG. 7, however the accelerometer 120 is instead used to output a signal corresponding to vibrations detected during a dose dispensing operation.

Optional steps 810 and 820 are the same as respective steps 710 and 720 of FIG. 7. Step 830 is the same as step 730 of FIG. 7.

At around the same time as step 830, in step 840, vibrations of the drug delivery device 200 are detected by the accelerometer 120, which outputs a corresponding signal.

In optional step 850, data corresponding to the signal output by the gyroscope 102 and the signal output by the accelerometer 120 is transmitted from the apparatus 100 to the computing device 410 via the communication interface 112. The signals output by the gyroscope 102 and the accelerometer 120 may have been at least partially processed by the processor 106 of the apparatus 100 to generate the data transmitted to the computing device 410.

In step 860, a dose dispensed by the drug delivery device 200 is determined based at least on the signal output by the gyroscope 120, in a similar manner as discussed previously. The dispensed dose may be determined by the computing device 410 or by the apparatus 100.

The dispensed dose may also be determined based on the signal output by the accelerometer 120. For example, the apparatus 100 or the computing device 410 may analyse the signal output by the accelerometer 120 to determine whether the vibrations or clicks detected by the accelerometer 120 are indicative of a dose dispensing operation being performed. If so, the dispensed dose may be calculated. If not, the dispensed dose may not be calculated, and instead the computing device 410 may transmit a signal to the apparatus 100 to place the apparatus in a low power mode (for example to stop the gyroscope 102 and accelerometer 120 taking further measurements).

If the vibrations or clicks detected by the accelerometer 120 are determined to be indicative of a dose dispensing operation being performed, the dispensed dose may be determined based on signals output from the gyroscope 102 while the accelerometer 120 was outputting a signal indication that a dose dispensing operation was taking place. In other words, the dispensed dose may be determined based on the signal output by the gyroscope 102 from when the accelerometer 120 started detecting vibrations indicative of the dispensing operation to when the accelerometer stopped detecting vibrations indicative of the dispensing operation.

Additionally or alternatively, in some examples the signal output by the accelerometer 120 may be used to determine a dispensed dose by determining the number of detected clicks and determining a dose based on the determined number of clicks, as discussed previously. The dose determined using the accelerometer 120 and the dose determined using the gyroscope 102 may both be used to determine a final dispensed dose, as discussed previously.

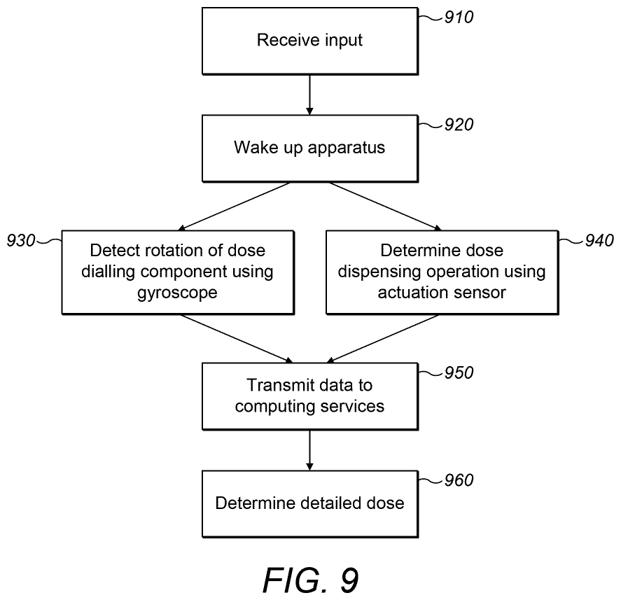

FIG. 9 shows a similar method to FIG. 7 and FIG. 8, however instead of using an accelerometer 102, an actuation sensor 130 is used to detect that a dose dispensing operation is being carried out.

Optional steps 910 and 920 are the same as respective steps 710 and 720 of FIG. 7. Step 930 is the same as step 730 of FIG. 7.

At around the same time as step 930, in step 940, actuation of part of the drug delivery device 200 by a user performing a dose dispensing operation is detected by an actuation sensor 130, which outputs a corresponding signal. The signal indicates that a dose dispensing operation is being performed by a user on the drug delivery device 200. The signal can be used to distinguish between actuation by a user and accidental actuation.

In optional step 950, data corresponding to the signal output by the gyroscope 102 and the signal output by the actuation sensor 130 is transmitted from the apparatus 100 to the computing device 410 via the communication interface 112. The signals output by the gyroscope 102 and the actuation sensor 130 may have been at least partially processed by the processor 106 of the apparatus 100 to generate the data transmitted to the computing device 410.

In step 960, a dose dispensed by the drug delivery device 200 is determined based at least on the signal output by the gyroscope 120, in a similar manner as discussed previously. The dispensed dose may be determined by the computing device 410 or by the apparatus 100. The signal output by the actuation sensor 130 may be used to confirm that a dose dispensing operation is taking place. The dispensed dose may be determined based on signals output from the gyroscope 102 while the actuation sensor 130 was outputting a signal indication that a dose dispensing operation was taking place. In other words, the dispensed dose may be determined based on the signal output by the gyroscope 102 from when the actuation sensor 130 started detecting the dose dispensing operation was taking place until when the actuation sensor 130 detected that the dose dispensing operation was no longer taking place.

In some examples, the steps of FIGS. 8 and 9 may be combined. For example, an accelerometer 120 may be used to detect vibrations, while an actuation sensor 130 may also be used to detect a user performing the dose dispensing operation. In this case, the actuation sensor 130 may provide the confirmation that a dose dispensing operation is taking place while the vibrations detected using the accelerometer 120 may be used to determine a dialled dose.

In any of the methods illustrated in FIGS. 7 to 9, the computing device 410 may determine that the dose dispensing operation has ended, for example based on the signals output by one or more of the gyroscope 102, accelerometer 120 and actuation sensor 130. In response to determining that the dose dispensing operation has ended, the computing device 410 may transmit a signal to the apparatus 100. The signal may cause the apparatus 100 to enter a low-power mode. For example, receipt of the signal by the apparatus 100 may cause the apparatus 100 to stop transmitting data to the computing device 410 and/or stop the gyroscope 102 and accelerometer 120 from outputting signals. This may conserve the power supply of the apparatus 100.

Transmitting data corresponding to the outputs of the gyroscope 102 and accelerometer 120 to the computing device 410 for further processing can mean that a smaller computational demand in placed on the processor 106 of the apparatus 100. However, in some cases some or all of the steps may be carried out by the processor 106 of the apparatus 100 and a separate computing device 410 may not be present.

The steps of FIGS. 6-9 may be performed in a different order to that shown in the respective Figures. In some cases, some of the steps of FIG. 6-9 may be omitted from the method, or additional steps may be performed.

The mode sensing arrangement 104 may perform a number of functions. Firstly, the mode sensing arrangement 104 may be used to determine whether a signal output by the gyroscope 102 is actually caused by a dose dialling or dose dispensing operation being performed on the drug delivery device 200, or whether it is instead caused by a different event, for example the drug delivery device 200 rolling about during transport of the drug delivery device 200. The mode sensing arrangement 104 can help distinguish between a dose dialling operation and a non-dose dialling operation and so therefore prevent a dialled dose being determined when a dose dialling operation is in fact not being performed. Likewise, the mode sensing arrangement 104 can help distinguish between a dose dispensing operation and a non-dose dispensing operation and so therefore prevent a dispensed dose being determined when a dose dispensing operation is in fact not being performed.

Secondly, if the mode sensing arrangement 104 comprises an accelerometer 120 then the accelerometer 120 may be used to determine a dialled dose independently of the gyroscope 102. The dialled dose determined using the accelerometer 120 can be compared with the dialled dose determined using the gyroscope 102 to determine a final dialled dose. For example, the final dialled dose may be an average of the dialled dose determined using the gyroscope 102 and the dialled dose determined using the accelerometer 120. In some cases the final dialled dose may be the higher of the dialled dose determined using the gyroscope 102 and the dialled dose determined using the accelerometer 120, or the lower of the dialled dose determined using the gyroscope 102 and the dialled dose determined using the accelerometer 120. By basing the final determined dialled dose on both the output of the gyroscope 102 and the output of the accelerometer 120, the accuracy and/or reliability of the final dose determination may be improved. For example, determining the dialled dose based on an output of the gyroscope 102 alone may not provide an accurate dialled dose determination if the drug delivery device 200 is rotated before the proper dose dialling operation has begun or after the operation has ended. Similarly, determining the dialled dose based only on clicks detected using the accelerometer 120 may not provide an accurate dialled dose determination because some of the clicks may not be detected during rapid rotation of the dose dialling component. By basing the final determined dialled dose on both the output of the gyroscope 102 and the output of the accelerometer 120, limitations of both methods of dose determination may be overcome.

While aspects of the present disclosure describe the drug delivery device 200 as an injection pen, it should be noted that the disclosure may also apply to other types of drug delivery device 200 such as patch pumps and infusion devices.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short-or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codeable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N-(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091 MAR-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrome.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen. Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present disclosure, which encompass such modifications and any and all equivalents thereof.

An example drug delivery device may involve a needle-based injection system as described in Table 1 of section 5.2 of ISO 11608-1:2014(E). As described in ISO 11608-1:2014(E), needle-based injection systems may be broadly distinguished into multi-dose container systems and single-dose (with partial or full evacuation) container systems. The container may be a replaceable container or an integrated non-replaceable container.

As further described in ISO 11608-1:2014(E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014(E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014(E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

Those of skill in the art will understand that modifications (additions and/or removals) of various components of the substances, formulations, apparatuses, methods, systems and embodiments described herein may be made without departing from the full scope and spirit of the present disclosure, which encompass such modifications and any and all equivalents thereof.

1.-18. (canceled)

19. A data collection apparatus for measuring a dose of medicament, the data collection apparatus comprising:
a gyroscope configured to detect rotation of a dose dialing component of a drug delivery device during at least one of a dose dialing operation or a dose dispensing operation and output a first signal corresponding to an amount of the rotation; and
a mode sensing arrangement configured to output a second signal indicative of whether the dose dialing operation or the dose dispensing operation is being performed on the drug delivery device.

20. The data collection apparatus according to claim 19, wherein the data collection apparatus is configured to determine the dose based at least in part on the first signal outputted by the gyroscope and the second signal outputted by the mode sensing arrangement.

21. The data collection apparatus according to claim 20, wherein the mode sensing arrangement comprises an accelerometer, and wherein the data collection apparatus is configured to determine the dose based on a first dose determined using the gyroscope and a second dose determined using the accelerometer.

22. The data collection apparatus according to claim 21, wherein the accelerometer is configured to detect vibrations of the dose dialing component.

23. The data collection apparatus according to claim 21, wherein at least one of the gyroscope or the accelerometer is a microelectromechanical system (MEM).

24. The data collection apparatus according to claim 19, wherein the mode sensing arrangement comprises an accelerometer configured to detect vibrations during the dose dialing operation or dose dispensing operation.

25. The data collection apparatus according to claim 22, wherein the data collection apparatus is configured to process a third signal outputted by the accelerometer to distinguish between a first type of vibration and a second type of vibration, wherein the first type of vibration corresponds to the dose dialing operation and the second type of vibration corresponds to the dose dispensing operation.

26. The data collection apparatus according to claim 19, further comprising a communication interface, wherein the data collection apparatus is configured to use the communication interface to transmit data corresponding to the first signal outputted by the gyroscope to a computing device for determining the dose.

27. The data collection apparatus according to claim 19, wherein the accelerometer is configured to detect linear translation of the dose dialing component during the dose dispensing operation.

28. The data collection apparatus according to claim 19, wherein the mode sensing arrangement comprises an actuation sensor configured to detect that the dose dispensing operation is being performed.

29. The data collection apparatus according to claim 28, wherein the actuation sensor comprises a capacitive touch sensor configured to detect a user's finger during the dose dispensing operation.

30. The data collection apparatus according to claim 19, wherein the drug delivery device is an injection pen.

31. The data collection apparatus according to claim 19, wherein the apparatus is contained within the drug delivery device.

32. The data collection apparatus according to claim 19, wherein the apparatus is a supplementary device attachable to the drug delivery device.

33. A system comprising:
a data collection apparatus for measuring a dose of medicament, the data collection apparatus comprising:
a gyroscope configured to detect rotation of a dose dialing component of a drug delivery device during at least one of a dose dialing operation or a dose dispensing operation and output a first signal corresponding to an amount of the rotation, and
a mode sensing arrangement configured to output a second signal indicative of whether the dose dialing operation or the dose dispensing operation is being performed on the drug delivery device; and
a computing device, wherein the computing device is configured to determine the dose based on the first signal outputted by the gyroscope and the second signal outputted by the mode sensing arrangement.

34. The system of claim 33, wherein the data collection apparatus comprises a communication interface, and wherein the data collection apparatus is configured to use the communication interface to transmit data corresponding to the first signal outputted by the gyroscope to a computing device for determining the dose.

35. A computer-implemented method for measuring a dose of medicament, the method comprising:
detecting, using a gyroscope, rotation of a dose dialing component of a drug delivery device;
determining, using a mode sensing arrangement, that a dose dialing operation or a dose dispensing operation is being performed on the drug delivery device; and
in response to determining that the dose dialing operation or the dose dispensing operation is being performed on the drug delivery device, determining a dose based on the rotation of the gyroscope.

36. The computer-implemented method according to claim 35, wherein determining that a dose dialing operation or a dose dispensing operation is being performed on the drug delivery device comprises:
detecting vibrations of the dose dialing component using an accelerometer of the mode sensing arrangement; and
determining that the detected vibrations are indicative of a dose dialing operation or a dose dispensing operation being performed on the drug delivery device.

37. The computer-implemented method according to claim 36, further comprising processing a third signal outputted by the accelerometer to distinguish between a first type of vibration and a second type of vibration, wherein the first type of vibration corresponds to the dose dialing operation and the second type of vibration corresponds to the dose dispensing operation.

38. The computer-implemented method according to claim 35, wherein determining that a dose dispensing operation is being performed on the drug delivery device comprises:
detecting actuation of the dose dialing component using an actuation sensor of the mode sensing arrangement.
